(19) United States
(12) Patent Application Publication
(10) Pub. No.: US 2011/0098234 A1
(43) Pub. Date: Apr. 28, 2011

(54) BREAST CANCER PROTEINS

(76) Inventors: Michael Cahill, Loerzweiler (DE); André Schrattenholz, Mainz (DE); Raffael Kurek, Dieburg (DE)

(21) Appl. No.: 11/575,228

(22) PCT Filed: Sep. 14, 2005
(86) PCT No.: PCT/EP2005/009871

§ 371 Date: Mar. 5, 2009

(30) Foreign Application Priority Data
Sep. 14, 2004 (EP) 04021801.8

(51) Int. Cl. A61K 38/17; A61P 35/00; C12Q 1/02

(52) U.S. Cl. 514/19.4; 514/19.3; 435/29

(57) ABSTRACT

The present invention relates to a pooling method for performing proteomics with human samples, preferably derived from microdissected human samples, wherein the samples are grouped into two or more pools and each pool reveals at least one protein that is differentially manifest between these pools but common to a member of a given pool. Besides, the invention refers to proteins whose abundances are upregulated in cancer cells, especially in breast cancer cells, preferably in breast cancer cells that are positive or negative towards hormone binding receptors.

The present invention relates to a method for performing proteomics, especially with human microdissected samples, the use of different proteins as diagnostic and/or therapeutic markers for breast cancer and the use of reagents to modulate aid proteins.

Breast cancer is one of the most common forms of cancer observed in women. With a predicted number of approximately 215.990 (32%) new cases and with over 40.000 deaths expected in the US in 2005. Consequently there is an increasing demand for a better understanding of molecular events underlying cancer in order to develop improved diagnostic and therapeutic avenues. Model systems, such as cell culture models, are often employed in an attempt to achieve this. However, much doubt exists as to how well results from such systems reflect the biology that is relevant in human disease, in particular cancer. Therefore, it is highly desirable to examine cells from primary clinical tissues. To this means gene array studies are commonly used because of the availability of suitable standard protocols. However, protein based phenomena, such as altered turnover or post-translational modifications, are not detected by gene arrays.

Proteomics is the study of proteins in complex biological systems. However, the methods associated with this field of science and known in the prior art are poorly suited for the analysis of most primary tissue samples of human origin, since the amounts of material that can be harvested are often extremely limited. Besides, the relevant disease cells are often mixed with many other cell types in tissues, requiring further techniques, in particular microdissection techniques, which can exacerbate the sample yield problem. The limited protein amounts can implicate the estimation of protein abundance with poor certainty. Thus, numerous replicates are required to achieve statistically robust results, which is furthermore a withdrawal concerning rare samples. A further problem is given by modern screening programs which detect tumours at an earlier and smaller stage.

Thus it is the object of the present invention to provide a method, markers, therapeutic targets and suited reagents for diagnosis and/or treatment of cancer, in particular breast cancer. With respect to the method it would be beneficial to provide a method that allows for a reliable estimation of protein abundance obtainable for instance from microdissection techniques applied on human samples.

To achieve this object, the invention firstly proposes a method having the features specified in claim 1. Further embodiments of the inventive methods are depicted in dependent claims 2 to 10. Additionally, the invention comprises the use of said method for performing proteomics and the use of several proteins as diagnostic markers and/or therapeutic targets as claimed in claims 11 to 20. Besides the invention relates to at least one reagent that modulates the expression and/or activity of at least one protein as claimed in claims 21 and 24. The wording of all claims is hereby incorporated in the description by reference.

The first object is achieved according to the invention by a method for performing proteomics with human samples, especially human microdissected samples, wherein the samples are grouped into two or more pools based on parameters of interest, said pools revealing at least one protein that is differentially manifest between these pools but common to members of a given pool.

The term “manifest” as used herein is equivalent to “abundant” both terms referring to the expression level of a protein being detectable with prior art methods, in particular with two dimensional so called ProteoTope analysis method of the applicant.

The term “tumour” as used herein refers to proliferative cells or tissues comprising all forms of benign or malignant properties or tendencies.

“Protein” as used according to the invention, comprises the entire or a partial sequence of the protein, wherein the partial sequence reveals the activity of the protein. Besides, the term “protein” comprises isoforms thereof.

Though, the benefits and disadvantages of a pooling strategy in general has been considered in other areas, particularly in the area of messenger RNA analysis (Kendziorski C M, Zhang Y, Lan H, Attie A D. 2003. The efficiency of pooling mRNA in microarray experiments. Biostatistics. 4:465-77.), the inventors have been the first to devise and test a pooling strategy with respect to protein phenomena, in particular for the purpose of diagnosing and/or treating cancer, especially breast cancer.

In an embodiment of the invention the human samples are pooled on the basis of being positive or negative towards at least one protein, particularly towards at least one receptor, preferably towards at least one hormone binding receptor, or mRNA thereof. Preferably, the receptor is the estrogen receptor (ER) and/or the progesterone receptor (PR).

The estrogen receptor (ER) comprises two types of specific nuclear receptors that are known as estrogen receptor α (ERα) and estrogen receptor β (ER(β). Molecular analysis has proven that ERα, like other nuclear receptors, consists of separable domains responsible for DNA binding (DNA binding domain DBD), hormone binding (hormone binding domain HBD) and transcriptional activation domain. The N-terminal activation function (AF-1) of the purified receptor is constitutively active, whereas the activation function located within the C-terminal part (AF-2) requires hormone for its activity.

ERα is found in 50-80% of breast tumours and ERα status is essential in making decisions about endocrine therapy with anti-estrogens, which are competitive inhibitors of endogenous estrogens and inhibit mitogenic activity of estrogens in breast cancer. On a molecular basis, they trigger inactive conformation of the ERα, which is then unable to activate transcription (Shiau A K, Barstad D, Loria P M, Cheng L, Kushner P J, Agard D A, Greene G L. 1998. The structural basis of estrogen receptor/coactivator recognition and the antagonism of this interaction by tamoxifen. Cell. 95:927-37.). The anti-estrogen tamoxifen is used to treat hormone-dependent breast cancers, where, after 5 years of treatment, it reduces disease recurrence and improves survival regardless of patient age and nodal status (Come S E, Buzdar A U, Arteaga C L, Brodie A M, Davidson N E, Dowsett M, Ingle J N, Johnston S R, Lee A V, Osborne C K, Pritchard K I, Vogel V G, Winer E P, Hart C S. 2003. Second international conference on recent advances and future directions in endocrine manipulation of breast cancer: summary consensus statement. Clin Cancer Res 9:443-446.). In patients with metastatic disease who have ER-positive tumours, tamoxifen is effective in approximately 50% of the cases (Fisher B, Jeong J, Dignam J, Anderson S, Mamounas E, Wickerham D L, and Wolmark N. 2001. Findings from recent National Surgical Adjuvant Breast and Bowel Project adjuvant studies in stage I breast cancer. J Natl Cancer Inst Monogr 30:62-66.).

In a preferred embodiment of the invention the estrogen receptor is a parameter of interest and the pools to be analysed are grouped on the basis of being positive or negative towards the estrogen receptor (ER+ vs. ER− pools). For instance, an appropriate pooling design is depicted in table 1. While the mitogenic action of estrogens in breast cancer is well established, there is evidence that estrogen receptors mediate protective, anti-invasive effects. Clinically, a positive ER status (ER+) correlates with favourable prognostic features, including a lower rate of cell proliferation and histologic evidence of tumour differentiation. In contrast to that, a negative ER status (ER−) corresponds to substantially poorer disease-free and overall survival probability of the patient. ER status is also prognostic for the site of gross metastatic spread. Besides, tumours with high abundance of estrogen receptor (ER+ tumours) are more likely to initially manifest clinically apparent metastasis in bone, soft tissue or the reproductive and genital tracks, whereas tumours with low abundance of Estrogen receptor (ER− tumours) more commonly metastasise to brain and liver. Several studies have correlated ERα expression to lower Matrigel invasiveness and reduced metastatic potential of breast cancer cell lines (Platet N, Prevostel C, Derocq D, Joubert D, Rochefort H, Garcia M. 1998. Breast cancer cell invasiveness: correlation with protein kinase C activity and differential regulation by phorbol ester in estrogen receptor-positive and -negative cells. Int. J. Cancer. 75:750-6, and Thompson E W, Paik S, Brunner N, Sommers C L, Zugmaier G, Clarke R, Shima T B, Torri J, Donahue S, Lippman M E, Martin G R, Dixon R B. 1992. Association of increased basement membrane invasiveness with absence of estrogen receptor and expression of vimentin in human breast cancer cell lines. J. Cell. Physiol. 150:534-544.).

Moreover, when ERα-positive cells are implanted in nude mice, tumours appear only in the presence of estrogens and are poorly metastatic as compared to those developed from ERα-negative (ERα−) breast cancer cell lines (Price J E, Polyzos A, Zhang R D, Daniels L M. 1990. Tumourigenicity and metastasis of human breast carcinoma cell lines in nude mice. Cancer Res. 50:717-721).

Furthermore, the invention encompasses the progesterone receptor (PR) as preferred parameter of interest in order to group the human samples, particularly obtained by microdissection techniques. For instance, an appropriate pooling design is depicted in table 5. Progesterone plays a major role in mammalian reproductive biology, including development of the normal mammary gland and expression of its differentiated function during pregnancy, and promotion of uterine differentiation and preparation for implantation in pregnancy. Progesterone effects are mediated via the progesterone receptor (PR), which like estrogen receptor (ER) is present 15% to 30% of luminal epithelial cells of the normal breast, and control of progesterone is largely although not exclusively achieved by control of the concentration of progesterone receptor (PR). Evidence for a role of progesterone in human breast development is provided by mouse models in which the progesterone receptor (PR) gene has been knocked out, which suggest that, whereas estradiol stimulates ductal elongation and PR expression, progesterone induces lobuloalveolar development.

According to a particularly preferred embodiment the human samples that are preferably obtained by microdissection techniques are grouped into two or more pools based on being positive or negative towards the estrogen receptor (ER) and being positive or negative towards the progesterone receptor (PR).

Preferably, the human samples, in particular obtained by microdissection techniques, are grouped into two or more pools based on being positive towards the estrogen receptor (ER+) and positive or negative towards the progesterone receptor (PR+ or PR−). For instance, an appropriate pooling design and a distribution to sample pools is depicted in table 10. As a result, the human samples are all positive towards the estrogen receptor (ER+), but differ in being either positive (PR+) or negative (PR−) towards the progesterone receptor (ER+/PR+ vs ER+/PR− pool). This is particularly beneficial since tumours expressing progesterone receptor in addition to estrogen receptor (ER+/PR+) significantly and independently correlate with increased propability of response to tamoxifen, whereas tumours expressing estrogen receptor but lacking progesterone receptor (ER+/PR−) are more responsive to the aromatase inhibitor Arimdiex than ER+/PR+ tumours.

Taken together, according to the invention it is possible to discriminate patients with ER+ tumours who respond to estrogen and/or anti-estrogens, in particular tamoxifen, from patients with ER+tumours who do not or less. Therefore the analysis of protein abundance (expression) of human samples based on ER+/PR+ and ER+/PR− pools as described above may contribute to improved individual therapy strategies.

In a further embodiment, the protein that is differentially manifest between the pools, preferably between pools based on being positive or negative towards the estrogen receptor (ER+ vs. ER−) is vimentin. It was found by the inventors that vimentin, particularly an isoform thereof, is significantly more abundant in cancer cells, especially in breast cancer cells, preferably in breast cancer cells that are negative for the estrogen receptor (ER−), or that have substantially poorer disease-free and overall survival probability.

In a further preferred embodiment of the invention, the protein is fibrinogen, particularly an isoform thereof, and/or fibrin, particularly non cross-linked fibrin, being significantly more abundant in cancer cells, especially in breast cancer cells, preferably in breast cancer cells that are negative for the estrogen receptor (ER−). As an alternative fibrinogen, particularly an isoform thereof, and/or fibrin, especially non cross-linked fibrin, are significantly more abundant in cancer cells, especially breast cancer cells, that have substantially poorer disease-free and overall survival probability. This association was surprisingly found by the inventors applying the inventive method on human samples that are derived from cancer tissue, especially from breast cancer tissue. The most highly differential manifest or abundant protein in ER− tumours relative to ER+ tumours due to the inventor's study is fibrinogen gamma A chain, followed by fibrin. Fibrinogen is proteolytically cleaved under the influence of platelets to produce a fibrin clot during the process of blood clotting. Cancer-related fibrin deposition and fibrinolysis characterizes many solid tumours, with cancer cells supplying many of the functions supplied by platelets in normal blood clotting (Gerner C, Steinkellner W, Holzmann K. Gsur A, Grimm R, Ensinger C, Obrist P, Sauermann G. 2001. Elevated plasma levels of cross-linked fibrinogen gamma-chain dimer indicate cancer-related fibrin deposition and fibrinolysis. Thromb Haemost. 85:494-50). The deposition of fibrinogen without subsequent conversion to fibrin in the tumour stroma is reportedly a hallmark of breast carcinoma. Endogenous synthesis and secretion of fibrinogen is, at least in part, the source of deposition in the extracellular matrix of breast cell carcinomas (Rybarczyk B J, Simpson-Haidaris P J. 2000. Fibrinogen assembly, secretion, and deposition into extracellular matrix by MCF-7 human breast carcinoma cells. Cancer Res. 60:2033-2039). Besides, it was found that estrogen destabilizes fibrinogen messenger RNA in frogs (Pastori R L, Moskaitis J E, Smith L H Jr, Schoenberg D R. 1990. Estrogen regulation of Xenopus laevis gamma-fibrinogen gene expression. Biochemistry 29:2599-605), and estradiol represses the fibrinogen gamma messenger RNA in fish (Bowman C J, Kroll K J, Gross T G, Denslow N D. 2002. Estradiol-induced gene expression in largemouth bass, Micropterus salmoides. Mol. Cell. Endocrinol. 196:67-77). In view of the down-regulation of the above mentioned proteins in ER+ tumours the negative effect of estrogen on the abundance (expression) of fibrinogen in ER+ cells might be phylogenetically conserved.

According to a further embodiment the protein is preferably XTP3-transactivated protein A. It was surprisingly found by the inventors that this protein is more abundant in cancer cells lacking the ER, especially in breast cancer cells, preferably in breast cancer cells that are negative for the estrogen receptor (ER−), or that have substantially poorer disease-free and overall survival probability.

Hpr6.6 protein, particularly two isoforms thereof, has been surprisingly found by the inventors to be significantly more abundant in cancer cells lacking the ER, especially in breast cancer cells, preferably in breast cancer cells that are negative for the estrogen receptor (ER−), or that have substantially poorer disease-free and overall survival probability. According to a preferred embodiment the isoforms of Hpr6.6 differ in their phosphorylation status. This protein is not related to the classical progesterone receptor. Hpr6.6 protein has a transmembrane domain N-terminally to a Cytochrome b₅ domain that does not interact with heme groups, but is rather thought to be involved in steroid binding. Such membrane-associated progesterone receptors are thought to mediate a number of rapid cellular effects not involving changes in gene expression. Obviously, they also have the potential to influence gene expression. Hpr6.6 protein regulates the responses to oxidative damage in the MCF-7 breast cancer cell line, leading to apoptosis.

In a further preferred embodiment of the invention the proteins are the retinoic acid-binding protein type II (RABP II), preferably cellular retinoic acid-binding protein type II (CRABP II), and/or secreted protein of un-known function (SPUF) that are significantly more abundant in cancer cells, especially in breast cancer cells, preferably in breast cancer cells that are positive for the estrogen receptor (ER+) and negative for the progesterone receptor (PR−), or as an alternative are significantly expressed in cancer cells, especially breast in cancer cells, that have substantially poorer disease-free and overall survival probability.

In addition, the inventors surprisingly found that the protein cytochrome b₅ is significantly more abundant in cancer cells, especially in breast cancer cells, preferably in breast cancer cells that are positive for the estrogen receptor (ER+) and positive for the progesterone receptor (PR+), or that have substantially better disease-free and overall survival probability. Cytochrome-b₅ is a heme-binding protein which is classically involved in the introduction of double bonds into long-chain acyl coenzyme A molecules to generate unsaturated fatty acids, and positively regulates some reactions catalysed by Cytochrome P450 proteins by acting as primary or secondary electron donor.

Another potential mechanism of involvement of cytochrome-b₅, and other differentially expressed proteins, in particular glutathione S-transferase and hemoglobin, concerns the heme iron electron transfer-related metabolism and detoxification of drugs and xenobiotic agents. Most potential human mammary carcinogens require multiple-enzyme-catalyzed steps to effect biotransformation into mutagenic metabolites. This may proceed through a primary metabolic step carried out in the liver, or through complete metabolic activation in the breast. Determining the relative contributions of hepatic and mammary carcinogenic activation is an important yet outstanding task. In the breast, several carcinogen-metabolizing enzymes are expressed, among which are multiple P450 proteins that may either activate (metabolise substrates to more reactive species) or detoxify (reduce the DNA reactivity of genotoxic species and/or increase their excretion). The majority of P450 proteins have multiple substrates, and some human cytochrome P450 proteins modify steroid hormons and oxidize numerous drugs (Nebert, D. W. & Russell, D. W. Clinical importance of the cytochromes P450. Lancet 360, 1155-1162. (2002); and Werck-Reichhart, D. & Feyereisen, R. cytochromes P450: a success story. Genome Biol 1, REVIEWS3003. Epub 2000 December 3008. (2000)).

According to a further embodiment of the inventive method the human samples are derived from a small tissue fraction, particularly from a tumour tissue fraction, advantageously from a breast cancer tissue fraction.

Furthermore, the invention comprises the use of a pooling strategy, in particular according to the aforementioned embodiments, for performing proteomics, especially with human samples, especially with human microdissected samples, wherein the samples are grouped into two or more pools, in particular of two or more samples per pool, based on parameters of interest, said pools revealing at least one protein that is differentially manifest between these pools but common to members of a given pool. Concerning further details it is referred to the above description.

The invention also encompasses the use of at least one protein that is derived from human samples, in particular microdissected human samples, as diagnostic marker and/or therapeutic target for cancer, especially breast cancer, that is associated with substantially poorer or better disease-free and overall survival probability, wherein the samples are grouped into two or more pools based on parameters of interest, said pools revealing said protein that is differentially manifest between these pools but common to members of a given pool. Preferably, the protein is selected from the group consisting of vimentin, fibrinogen, fibrin, especially non-cross-linked fibrin, XTP3-transactivated protein A, retinoic acid-binding protein type II (RABP II), especially cellular retinoic acid-binding protein type II (CRABP II), cytochrome b₅, secreted protein of unknown function (SPUF) and progesterone receptor membrane component 1 (Hpr6.6 protein).

Preferably, vimentin, in particular an isoform thereof, whose abundance is significantly upregulated in cancer cells, especially in breast cancer cells, preferably in breast cancer cells that are negative for the estrogen receptor (ER−), is used as diagnostic marker and/or therapeutic target for cancer, especially breast cancer, that is associated with substantially poorer disease-free and overall survival probability.

In another embodiment of the invention fibrinogen, especially at least one isoform thereof, and/or fibrin, especially non cross-linked fibrin, whose abundance is significantly increased in cancer cells, especially in breast cancer cells, preferably in breast cancer cells that are negative for the estrogen receptor (ER−) are used as diagnostic markers and/or therapeutic targets for cancer, especially breast cancer, that is associated with substantially poorer disease-free and overall survival probability.

A further protein that is preferably used as diagnostic marker and/or therapeutic target for cancer, especially breast cancer, that is associated with substantially poorer disease-free and overall survival probability is XTP3-transactivated protein A, whose abundance is significantly increased in cancer cells, especially in breast cancer cells, preferably in breast cancer cells that are negative for the estrogen receptor (ER−).

In an additional embodiment of the invention retinoic acid-binding protein type II (RABP II), especially cellular retinoic acid-binding type II (CRABP II), whose abundance is significantly increased in cancer cells, especially in breast cancer cells, preferably in breast cancer cells that are positive for the estrogen receptor (ER+) and negative for the progesterone receptor (PR−), is used as diagnostic marker and/or therapeutic target for cancer, especially breast cancer, that is associated with substantially poorer disease-free and overall survival probability.

In a further aspect of the invention, cytochrome b₅, whose abundance is significantly increased in cancer cells, especially in breast cancer cells, preferably in breast cancer cells that are positive for the estrogen receptor (ER+) and positive for the progesterone receptor (PR+), is preferably used as diagnostic marker and/or therapeutic target for cancer, especially breast cancer, that is associated with substantially better disease-free and overall survival probability.

According to a further embodiment of the invention, secreted protein of unknown function (SPUF) whose abundance is significantly increased in cancer cells, especially in breast cancer cells, preferably in breast cancer cells that are positive for the estrogen receptor (ER+) and negative for the progesterone receptor (PR−), is used as diagnostic marker and/or therapeutic target for cancer, especially breast cancer, that is associated with substantially poorer disease-free and overall survival probability.

The invention also encompasses the use of at least one of three isoforms of progesterone receptor membrane component 1 (Hpr 6.6 protein), whose abundance is significantly increased in cancer cells, especially in breast cancer cells, preferably in breast cancer cells that are negative for the estrogen receptor (ER−), as diagnostic marker and/or therapeutic target for cancer, especially breast cancer, that is associated with substantially poorer disease-free and overall survival probability.

In a further embodiment of the invention it is beneficial to use a combination of proteins, in particular of the aforementioned proteins or isoforms thereof, as diagnostic markers and/or therapeutic targets for cancer, especially breast cancer, that is associated with substantially poorer or better disease-free and overall survival probability, wherein the samples are grouped into two or more pools based on parameters of interest, said pools revealing said proteins that are differentially manifest between these pools but common to members of a given pool.

In another aspect of the present invention, it can be advantageous to use some or all of the relative protein abundances depicted at least in one table selected from table 2, table 6, table 8 and table 9 as diagnostic markers and/or therapeutic targets for cancer, especially breast cancer, that is associated with substantially poorer or better disease-free and overall survival probability. Results of protein identification are depicted in tables 3 and 7.

Finally, the invention encompasses the use of at least one reagent that modulates the abundance and/or activity of at least one protein or isoform thereof, derived from human samples, preferably from human microdissected samples, for the manufacture of a medicament for diagnosis and/or therapy of cancer, especially breast cancer, that is associated with substantially poorer or better disease-free and overall survival probability, wherein the samples are grouped into two or more pools based on parameters of interest, said pools revealing said protein that is differentially manifest between these pools but common to members of a given pool.

The invention further encompasses the use of at least one reagent that modulates the abundance and/or activity of at least one protein or isoform thereof, derived from human samples, preferably human microdissected samples, for diagnosis and/or therapy of cancer, especially breast cancer, that is associated with substantially poorer or better disease-free and overall survival probability, wherein the samples are grouped into two or more pools based on parameters of interest, said pools revealing said protein that is differentially manifest between these pools but common to members of a given pool.

Preferably, the pools of the last two embodiments are based on the parameter being positive or negative towards the estrogen receptor (ER+ vs. ER−). According to a more preferred embodiment of the invention, the pools are based on the parameters being positive towards the estrogen receptor and positive or negative towards the progesterone receptor (ER+/PR+ vs. ER+/PR−). This pool strategy is especially advantageous, since it allows for the discrimination of cancer patients, in particular breast cancer patients, that are positive for the estrogen receptor but have different prognostic features concerning the success of a therapy, in particular a hormonal therapy. With respect to the proteins and/or isoforms thereof, whose abundances and/or activities are to be modulated is referred to the above description. Preferably, the protein whose abundance and/or activity is to be modulated is at least one member of the group consisting of XTP3-transactivated protein A, retinoic acid-binding protein type II (RABP II), especially cellular retinoic acid-binding protein type II (CRABP-II), cytochrome b₅ (Cyt-b₅), secreted protein of unknown function (SPUF) and progesterone receptor membrane component 1 (Hpr6.6 protein).

In a further particularly preferred embodiment of the invention the reagent modulates the activity of the protein, in particular the activity of at least one of the aforementioned proteins, preferably the activity of Hpr6.6 or at least one isoform thereof, by influencing, in particular increasing or decreasing, the degree of phosphorylation of the protein.

The tables and drawings show:

Table 1: Pooling design for experiment: ER+/ER−.

Table 2: Protein spot quantification for breast cancer samples (pool tumour slices) comparing ER+ and ER− samples. The spot fraction for ER+ and ER− with standard error is given as percent of the total spot volume (ER+ plus ER−); these values have been estimated using a least square fit for a model based on all replicates and accounting for patient pools as random effect; the p-value for this model is also given; spots with p-value<0.01 are bold. Spot number 18 has p>0.05, yet is included because it is the same gene product as spots 28 and 31.

Table 3: Details of identification of proteins from Table 2. No=spot number from Table 2. PI=Isoelectric Point. MW=Molecular Weight. Exp.=Experimentally observed values. AccNo=Accession Number.

Table 4: Proteins identified in Table 2 and Table 3 and for which more than one protein spot isoform was identified in 2D-PAGE gels of ER+ vs. ER− breast cancer tumours. Each designated spot was observed with different experimental PI and MW. Labelling follows Table 3.

Table 5: Pooling design for experiment: PR+ vs. PR−, microdissected.

Table 6: Protein spots quantification for breast cancer samples comparing PR+ and PR− samples from; the spot fraction for PR+ and ER+/PR− with standard error is given in percent of the total spot volume (ER+ plus ER−); these values have been estimated using a least square fit for a model based on all replicates and sample pools; the p-value for this model is also given; in general spots have selected if the p-value was less than 0.01.

Table 7: Mass spectrometric identification of all spots from ER+/PR+ vs. ER+/PR− tumours that are congenic with spots from Table 6.

Table 8: Protein spot quantification and identifications for breast cancer samples (whole tumour slices) comparing ER positive and ER negative samples. N.i.=not identified. Gene Bank Identifies are from the NCBI data base version of Apr. 4, 2004. MALDI-TOF peptide mass fingerprinting (PMF) scores are from MASCOT. The average spot fraction for ER⁺ and ER⁻ are given as percent of the normalised total spot volume for each spot (=(ER⁺×100%)/(ER⁺+ER⁻)) across all patient pools based on two colour ProteoTope analysis for the indicated most significant protein spots. These values were obtained using a least square fit for a model based on all replicates and attributing pool variability as a random effect. The t-test p-value for this model is also given. P-values<0.001 are bold, and p-value<0.001 are designated as such. The bars at the right depict average percent abundance of each protein across the ER⁺ (dark blue) and ER⁻ (light orange) pools as indicated above the column with bars (0%-50%-100%). Error bars show standard error of means. Protein spots between numbers 37 and 38 (indicated by a grey field) are not presented, having failed to meet selection criteria of either abundance difference ratio of 1.5 or significance at the 5% level.

Table 9: Protein spots quantification for breast cancer samples comparing PR positive and PR negative samples from LCM, showing spots that differed significantly by more than 1.5-fold on average. The spot fraction for ER⁺/PR⁺ (darker blue) and ER⁺/PR⁻ (lighter orange) with standard error is given in percent of the total spot volume (PR⁺+PR⁻). N.i.=not identified. Gene Bank Identities are from the NCBI data base version of Apr. 4, 2004, MALDI-TOF peptide mass fingerprinting (PMF) scores are from MASCOT. The average spot fraction for ER⁺/PR⁻ (lighter orange) and ER⁺/PR⁺ (darker blue) are given as percent of the normalised total spot volume for each spot (e.g.=(PR⁺×100%)(PR⁺+PR⁻)) across all patient pools based on two colour ProteoTope analysis for the indicated most significant protein spots. These values were obtained using a least square fit for a model based on all replicates and attributing pool variability as a random effect. The t-test p-value for this model is also given. P-values<0.001 are bold, and p-values<0.001 are designated as such. The bars at the right depict average percent abundance of each protein across the ER⁺ (dark/blue) and ER⁻ (light/orange) pools as indicated above with bars (0%-50%-100%). Error bars show standard errors of means. Protein spots between 2-18 and 1-05 (indicated by a grey field) are not presented, having failed to meet selection criteria of either average abundance difference ratio of 1.5 (60%:40%) or significance at the 5% level.

Table 10: Samples used in this study, and distribution to sample pools. Twelve ER+/PR− and twelve ER+/PR+ tumours are grouped into four pools of three tumours each as indicated. All tumours were isolated with 10,000 laser shots (laser beam: 30 μm in diameter. Clinical data comprise: tumour status ranging from Tp1 (tumour 2 cm or smaller in greatest dimension) to pT3 (tumour>5 cm); lymph node status from pN0 (no regional lymph node metastasis) to pN3 (metastasis to ipsilateral internal mammary lymph nodes) and pNx (regional lymph node cannot be assessed); tumour grade from 2 (moderately differentiated) to 3 (poorly differentiated); histopathological data for ER and PR (0: undetectable, 1-3 weakly positive, 4-7: moderately positive, 8-12: highly positive); and HER2/neu-status (0=negative, positive +1 to +3). The number of 10 μm whole tumour slices provided for preparative protein identification from each tumour, and the total amount of protein from all combined preparative slices for each test condition are also provided in the right hand columns.

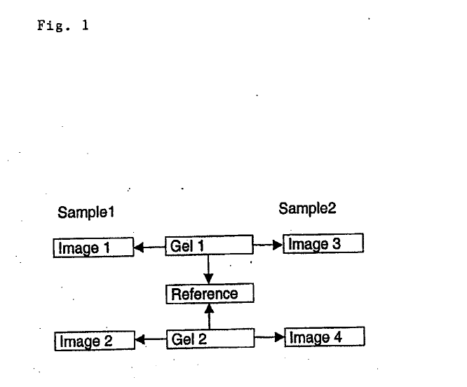

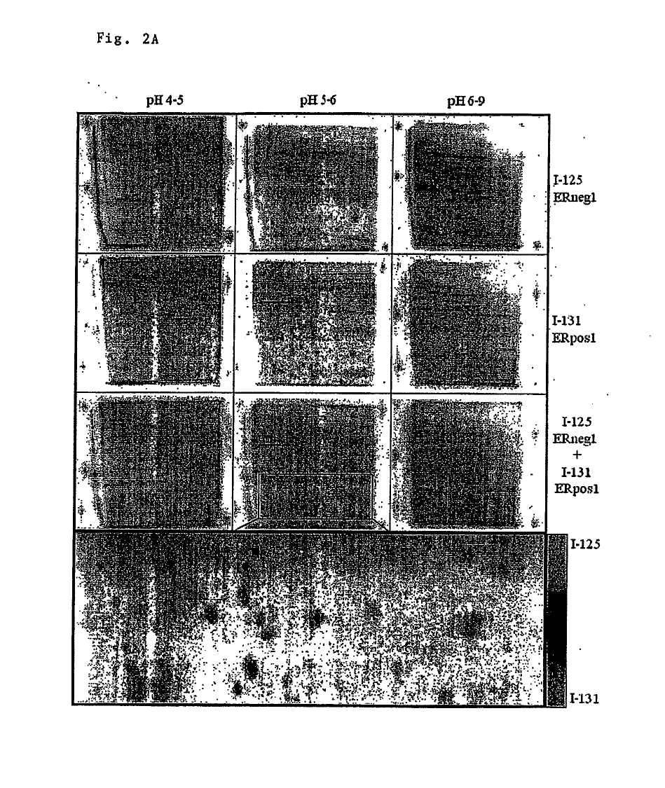

FIG. 1: Analysis scheme for ProteoTope gels: from each gel two images can be extracted using the ProteoTope technology, one for each two samples. Image 1 and image 2 differ only in the isotope used for labelling sample 1 and likewise image 3 and image 4 are images that can be extracted for sample 2.

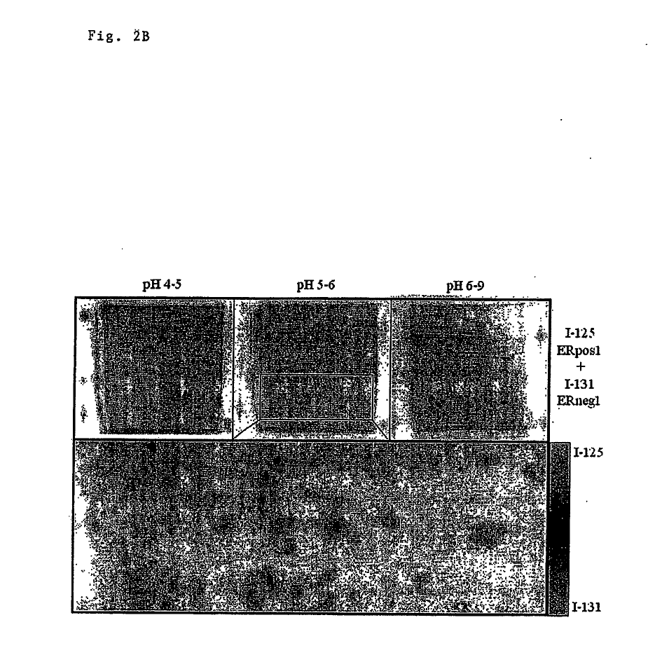

FIG. 2: 1.54 cm differential ProteoTope analysis. The panels show actual images from an inverse replicate labelled ProteoTope experiment for one sample pair. A. Analysis of pooled sample ER+1 (Erpos1) from Table 12 labelled with I-125, differentially compared with pooled sample ER-1 (ERneg1) labelled with I-131. The lower panels show the signal detected for each isotope, depicted in false spectral colour. The signals for each isotope have been normalised against each other for total relative intensity in the upper dual channel images, where the signal for I-125 is blue, the signal for I-131 is orange, and equal amounts of both signals produces grey or black signal. Two pure sources each of I-131 and I-125, as well as a 50% mixture of both isotopes, are measured on round 2 mm pieces of filter paper placed next to each gel as imaging controls. Cross talk between the signals from each isotope is <1%. The pH ranges of the 18 cm IPGs (Immobilised pH Gradients) used for serial IEF are indicated above the panels, and the radioactive iodine isotope signals depicted in each panel are indicated on the right. In this experiment all iodination reactions were performed on 60 μg protein. In the examples shown, the I-125 signal is systematically stronger in all gels (compare lower panels for individual isotopes). B. The top panels show the inverse replicate experiment of A, where sample ER+1 is labelled with I-131, and sample ER-1 is labelled with I-125. The bottom panel shows an enlarged portion of a gel image, as indicated. Similar gels were produced for all corresponding differential analyses depicted in Table 1.

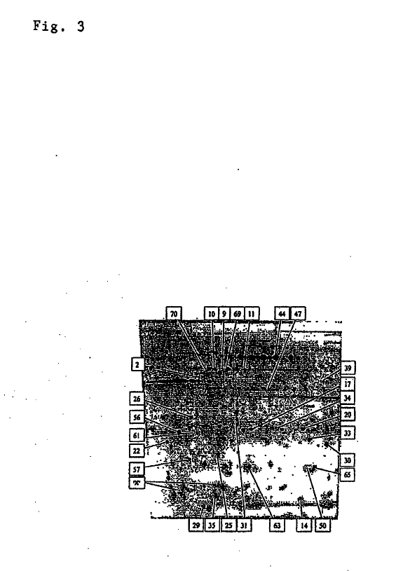

FIG. 3: Typical example of a synthetic average composite gel of the pH 5-6 analysis, showing spots matched across all gels in the study in this pH range from Table 8. The average ER+ signal is indicated as blue, the average ER− signal is indicated as orange, and equal intensities of both signals give grey or black pixels. Spot numbers correspond to Table 8. Some orange or blue spots that are not numbered (e.g., those labelled “X”) were not visible on preparative silver stained tracer gels, and were omitted from the analysis. This image was generated with the GREG software. Labels were added manually.

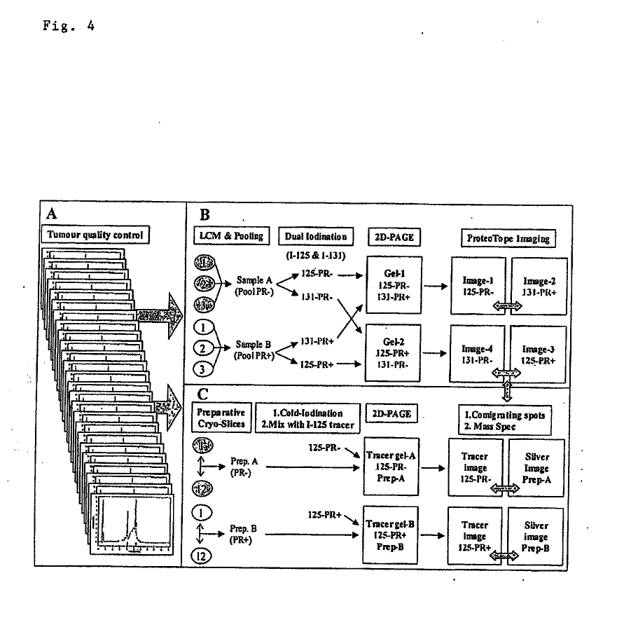

FIG. 4: Experimental ProteoTope Inverse Replicate and Tracer Gel Design. A) The quality of RNA from all tumours is first verified by Agilent Bioanalyzer. Only samples with sharp 18S and 28S ribosomal RNA are used. B) Analytical ProteoTope multiplex for the differential analysis of one pair of LCM sample pools. Samples from 3 separate LCM-harvested ER+/PR− (PR−) or ER+/PR+ (PR+) tumours are respectively mixed, and aliquots of the pooled sample are separately iodinated with each of two radioactive iodine isotopes: I-125 (blue) and I-131 (orange). The radioactively labelled proteins are mixed together and coelectrophoresed on inversely labelled replicate 2D-PAGE gels as shown. The individual signals from each radioisotope are measured by ProteoTope and mutually calibrated. Double headed arrows represent multiple image analysis. This study involved four such pools of three tumours each for each condition (PR−, PR+), and four paired analyses of the depicted design. C) Preparative amounts of protein were obtained from 35-45 different cryogenic slices (Table 10) from all 12 of the same tumours for each condition that were used for LCM, and pooled into PR− and PR+ pools as shown. Preparative pooled proteins are separately cold-iodinated, mixed with a trace of radiolabelled protein from a pool of all four sample ¹²⁵I-labelled pools corresponding to those of B and coelectrophoresed by 2D-PAGE. The radioactive 2D-PAGE image is matched to images from B, and silver-stained spots comigrating with radioactively labelled proteins of interest in the tracer gels are identified by mass spectrometry. The present study involved the production tracer gels corresponding to a duplicate of the depicted design.

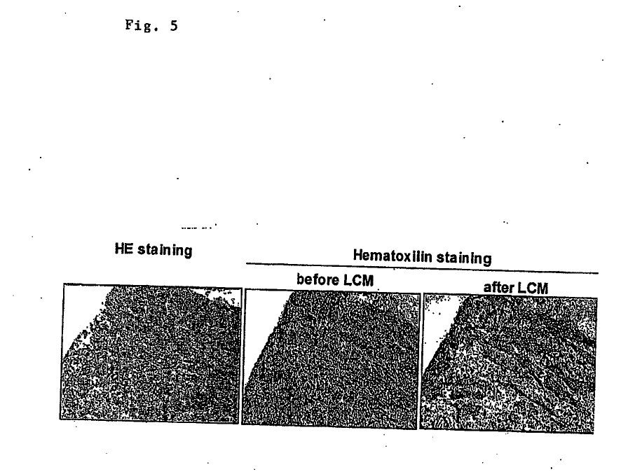

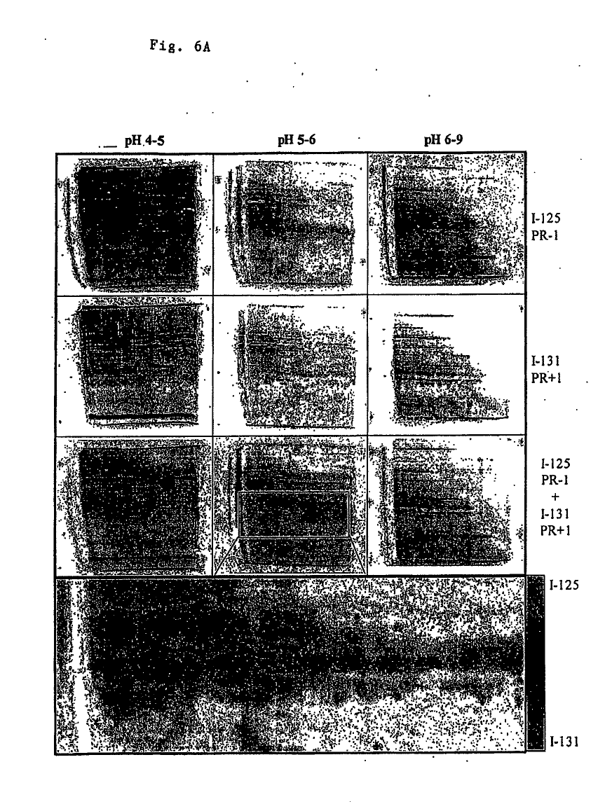

FIG. 5: Cancer cells isolated by laser microdissection. A frozen section of breast cancer tissue was stained with conventional hematoxylineosin (HE) staining (left panel). The neighbouring section was stained with hemtoxylin (middle panel) and the tumour cells were recovered from that section by means of laser capture microdissection (right panel).

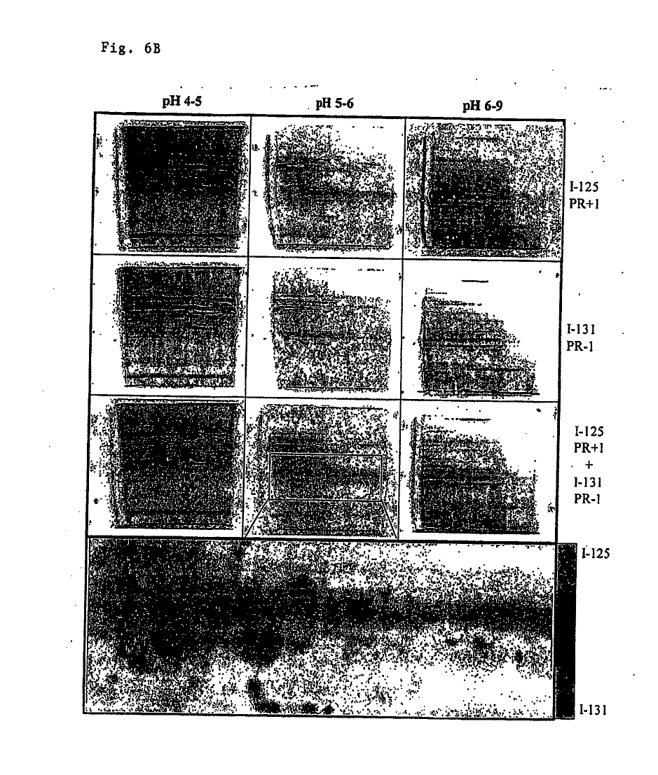

FIG. 6: 54 cm daisy chain IEF (isoelectric focusing) differential ProteoTope analysis of pooled LCM (Laser Capture Microdissection) samples. The panels show actual images from an inverse replicate labelled ProteoTope experiment for one pooled sample pair.

A) Analysis of pooled sample PR-1 (ER⁺/PR⁻ pool 1) labelled with I-125, differentially compared with pooled sample PR+1 (ER⁺/PR⁺ pool 2) labelled with I-131. The upper panels show the signal detected for each isotope, depicted in false spectral colour. The signals for each isotope have been normalised against each other for total relative intensity in the lower dual channel images, where the signal for I-125 is blue, the signal for I-131 is orange, and equal amounts of both signals produces grey or black signal. Two pure sources each of I-131 and I-125, as well as a 50% mixture of both isotopes, are measured on round 2 mm pieces of filter paper placed next to each gel as imaging controls. Cross talk between the signals from each isotope is <1%. The pH ranges of the 18 cm IPGs (Isoelectric Proteo Gels) used for serial IEF are indicated above the panels, and the radioactive iodine isotope signals depicted in each panel are indicated on the right. In this experiment, approximately 180 ng of protein from each pooled sample was loaded to each gel, and the above result was obtained by labelling approximately 3.6 μg of protein from each pooled sample. B) The top panels show the inverse replicate experiment of A, where pooled sample PR-1 is labelled with I-131, and pooled sample PR+1 is labelled with I-125. Similar gels were produced for all corresponding pooled sample pairs 1-4.

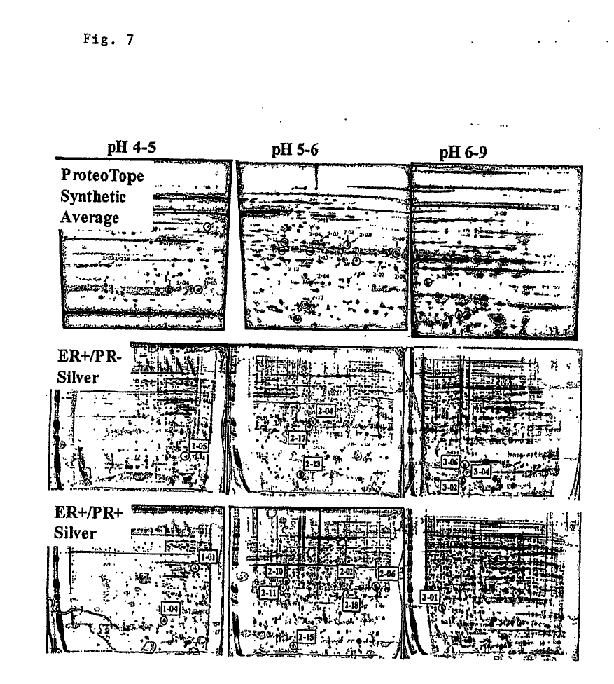

FIG. 7: Synthetic average and preparative Tracer gels for protein identification. The top panel shows a synthetic average daisy chain ProteoTope gel showing ER⁺/PR⁻ (blue) and ER⁺/PR⁺ (orange) spots detected across all gels from FIG. 3 for each pH range. The MALDI-TOF-MS PMF identification and log-odds statistical significances of red-circled spots are given in FIG. 6. The lower two panels show the position of these differentially identified proteins on silver stained preparative tracer gels loaded with 240 μg cold-labelled protein from ER⁺/PR⁻ (center panel) or ER⁺/PR⁺ (lower panel) tissue. The silver-stained gels, which are not used for quantification, were optically scanned with a plastic film covering, which is visible in some images.

Further advantages, features and possible uses of the invention are described below by means of the exemplary embodiments with reference to the above described tables and figures. In this connection, the various features may in each case be implemented on their own or in combination with one another.

Materials and Methods

Patients and tissue samples. Primary breast cancer specimens were obtained with informed consent from patients, who were treated at the Department of Gynecology and Obstetrics, University Hospital Tübingen (Ethikkommission Med. Fakultät AZ.266/98). Samples were characterized and collected by an experienced pathologist. After removal of breast tumour from the patient, the tissue samples were embedded in O.C.T. compound (Leica), then snap frozen in liquid nitrogen within 15 minutes of tumour removal, and stored at −196° C. in a tumour tissue bank. Sample collection was approved by an ethics committee and by the patient. Tumour data were stored in an Oracle-based database according to practices approved by the Institute of Electrical and Electronics Standards Association (IEEE-SA). Clinical information was obtained from medical records and each tumour was diagnosed by a pathologist, according to histopathological subtype and grade. The tissue quality of each tumour was verified by measuring RNA integrity from one or more slices with an Agilent 2001 Bioanalyser. Tumours lacking sharply distinct 18S and 28S ribosomal RNA bands were excluded from the study. ER, PR and HER-2/neu status for each tumour were routinely determined by immunohistochemistry. HER (Heregulin)/neu (Neuregulin) states of +2 and +3 (Table 10) were additionally confirmed by fluorescence in situ hybridisation. Clinical and pathological information and clinical data for each patient are summarized in Table 10.

Preparation of Cryosections.

Tumour samples were selected using the database, removed from the tissue bank on frozen CO₂ and transferred to a cryotome (Leica) at a temperature of −23° C. Cryogenic sections (10 μm) were subsequently sliced, placed on SuperFrost+-slides (Multimed) and stored at −80° C. until further use. For immunopathologic characterisation by an experienced pathologist one section was stained with hematoxilin/eosin and tumour cells suitable for laser microdissection were identified.

Two Dimensional ProteoTope analysis

2D-PAGE was performed using 18 cm commercial IPGs of pH 4-7 (Amersham) and 20 cm SDS-PAGE gels (Hoefer ISO-DALT), or own construction pH 4-9 IPGs of 54 cm that were run in the SDS-PAGE dimension as 3×18 cm IPGs in an ISO-DALT as described (Poznanovic S, Schwall G, Zengerling H, Cahill M A, 2005. Isoelectric focusing in serial immobilized pH gradient gels to improve protein separation in proteomic analysis. Electrophoresis. 26: 3185-90; The ProteoSys proprietary ProteoTope platform (Cahill M A., Wozny W, Schwall G, Schroer K, Holzer K, Poznanovic S, Hunzinger C, Vogt J A, Stegmann W, Matthies H, Schrattenholz A. 2003. Analysis of relative isotopologue abundances for quantitative profiling of complex protein mixtures labelled with the acrylamide/D3-acrylamide alkylation tag system. Rapid Communications in Mass Spectrometry, 2003, 17:1283-1290.) is a combination of methods developed by ProteoSys AG, including radio-iodination, 2D-PAGE, and high sensitivity radio-imaging. ProteoTope includes proprietary radio-imaging, which can discriminate between ¹²⁵I and ¹³¹I signals in one 2D-PAGE gel to generate a quantitative multicolour differential display of proteins from separate samples labelled with different iodine isotopes.

ProteoTope gels are produced for quantification, since the ProteoTope technology allows the most accurate quantification. Because the signal is radioactive, error is proportional to the square root of the number of detected signals. For all but the weakest signals, the measurement errors are negligible for subsequent differential quantification. No image registration (spot alignment) of the two sample images acquired from one gel is necessary. Thus a direct comparison of integrated spot intensities for the samples run on one gel can be used for further analysis. A synthetic reference must be introduced in order to compare the image pairs in gel 1 with those on gel 2 (see FIG. 1).

Since for ProteoTope gels the two samples to compare are run on one gel the expression analysis does not suffer from inter gel variations as silver stained gel analyses do. The major source of error is a potential bias towards the labelling stoichiometry of one or the other isotope. This can be monitored by producing the gels in inverse matched cross labelled duplicate fashion. When the images of one sample labelled with each of two isotopes do not correspond, quality criteria are not met and the analysis is repeated.

For ProteoTope gels a dual colour overlay can be generated with some computational effort to highlight differences between the two samples. Consistent differences between the samples will appear in both images in alternate channels.

Tracer Control Enrichment Gels

Analytical two colour ProteoTope gels are loaded with too little sample (<1 μg protein) to permit protein identifications with current methods. For preparative protein identification, tracer control enrichment gels (tracer gels) are employed, where a trace of radioactively labelled protein sample, corresponding to the sample used for analytical two colour gels, is co-electrophoresed with a vast excess of non-radioactively labelled protein (about 200 μg) to provide preparative amounts of protein for identification. Overlaying the silver stained gel images and the ProteoTope radioactive gel images permits the location of the position of ProteoTope-detected proteins on gels that are silver stained for spot picking. These images are not used for quantification. On each gel 200 μg of protein is applied and electrophoresis is performed as described before for ProteoTope gels (Poznanovic S, Schwall G, Zengerling H, Cahill M A. 2005. Isoelectric focusing in serial immobilized pH gradient gels to improve protein separation in proteomic analysis. Electrophoresis. 26: 3185-90; Cahill M A, Wozny W, Schwall G, Schroer K, Holzer K, Poznanovic S, Hunzinger C, Vogt J A, Stegmann W, Matthies H, Schrattenholz A. 2003. Analysis of relative isotopologue abundances for quantitative profiling of complex protein mixtures labelled with the acrylamide/D3-acrylamide alkylation tag system. Rapid Communications in Mass Spectrometry, 2003, 17:1283-1290). After electrophoresis gels are silver stained according to Shevchenko et al. (Shevchenko A, Wilm M, Vorm O, Mann M. 1996. Mass spectrometric sequencing of proteins silver-stained polyacrylamide gels. Analytical Chemistry. 1996; 68:850-858).

Image Analysis

The differential protein expression analysis is based on the reliable differential quantification of the protein spots on ProteoTope gels. For the quantitative image analysis it was employed the Phoretix 2D Advanced software (Nonlinear Dynamics), Delta 2D (Decodon GmbH) and GREG (Fraunhofer-Institut für Angewandte Informationstechnik). ProteoSys has added tools for automated data extraction, statistical analysis, and report generation.

Image Quantification Protocol

The analysis of ProteoTope and silver stained gels is typically performed according to the following protocol (e.g. for Phoretix 2D):

Detecting Spots for Each Image

Calibration of image intensity to counts (for ProteoTope images only) Automatic spot detection with manual adjustment of detection parameters (for Phoretix 2D these parameters are called sensitivity, noise, operator size, and background)

Manual splitting or merging of spots when needed

Background subtraction with mode of non-spot which uses a number of pixels around each spot that are not part of any other spot to determine the background

Matching the Images Against a Reference Gel

Manually defining user seeds to give an initial set of matching spots

Automatic matching of all spots in all images against the reference gel

Manual correction to unmatched spots if needed

Spot Selection

Spot selection is based on quantitative criteria as well as gel image quality and spot detection quality. For statistical calculations the logarithm to base 2 of the intensity ratio of the two samples applied on the gel is used i.e. log₂(Iₛₐₘₚₗₑ₁/Iₛₐₘₚₗₑ₂). The resulting quantity is usually called logodd. The mean values and standard errors are modeled from the logodd values taking advantage of the gel replicates and using the patients as random effect. This allows calculation of p-values using a t-test that is based on the model. If there is good evidence from the gel images that one of the spot intensities is an outlier, e.g. stripes or other distortions occur, then this one spot's intensity may be discarded. If a spot is not detected on one image its intensity is set to zero for that image unless it can clearly be identified as an outlier, in which case it will be discarded.

In general a spot is selected for further analysis if the p-value is lower than 1% and the absolute logodd value is higher than 0.6 for ProteoTope corresponding to a ratio of 1.5. At that point the image quality is checked to assure that the spot was consistently detected on all images. Additional spots may be selected even if the p-value is less higher than 1%: e.g. if the expression ratio is high, if the spot is in the close vicinity of a significantly different spot, or if there are no or very few spots that are significantly different otherwise. In any case the table with the quantitative analysis is presented without modifications to allow for estimation how reliable a difference is from the statistical point of view. The ProteoTope inverse labelled cross replicate data structure resembles that of replicated microarray experiments, for which established statistical methods are available (Pan W. 2002. A comparative review of statistical methods for discovering differentially expressed genes in replicated microarray experiments. Bioinformatics. 18:546-554).

Protein Identification by Mass Spectrometry

Protein identification is based on different mass spectrometric methods: an automated procedure that allows a very quick and reliable identification of higher abundant proteins (peptide mass fiungerprinting with MALDI-TOF-MS) but also allows the identification of very low abundant proteins with more time consuming procedures (LC-ESI-IonTrap-MS/MS, or MALDI TOF-TOF). Briefly, gel plugs of selected protein spots are excised and the proteins contained in the gel plugs are digested using trypsin. The resulting solution is analysed first with a high throughput peptide mass fingerprint procedure based on MALDI-TOF-MS. For those spots where only ambiguous identification was achieved, a fragment ion analysis based on MALDI TOF-TOF or LC-ESI-IonTrap-MS/MS was added. A detailed description of typical MALDI-TOF-MS procedures has been published (Vogt J A, Schroer K, Holzer K, Hunzinger C, Klemm M, Biefang-Arndt K, Schillo S, Cahill M A, Schrattenholz A, Matthies H, Stegmann W. 2003. Protein abundance quantification in embryonic stem cells using incomplete metabolic labelling with 15N amino acids, matrix-assisted laser desorption/ionisation time-of-flight mass spectrometry, and analysis of relative isotopologue abundances of peptides. Rapid Communications in Mass Spectrometry, 2003;17:1273-1282).

Database Searching

For the identification of the proteins the peptide masses extracted from the mass spectra were searched against the NCBI non-redundant protein database (www.ncbi.nlm.nih.gov) using MASCOT software version 1.9 (Matrix Science, London, detailed description can be found at http://www.matrixscience.com/).

Laser Microdissection and Sample Pooling

Using a laser capture microscope (Arcturus-PixCell-IIe) tumour cells were isolated, transferred into tubes, and stored at −80° C. according to manufacturer's instructions, then transported in the presence of frozen CO_{2. 10,000} LCM shots were acquired from each tumour, using a 30 μm laser setting. Pools of lysed protein samples from three patients were made. Of the resulting 30,000 LCM shot equivalent per pool, 10,000 shot equivalents were labelled with ¹²⁵I, 10,000 shot equivalents were labelled with ¹³¹I, and 10,000 shot equivalents went towards the construction of a joint pool of all 12 patients for each condition. The latter joint pools were iodinated with ¹²⁵I and used as radioactive tracer in preparative gels.

Proteomics Analysis

ProteoTope analysis was performed essentially as described (Schrattenholz A, Wozny W, Klemm M, Schroer K, Stegmann W, Cahill M A. 2005. Differential and quantitative molecular analysis of ischemia complexity reduction by isotopic labelling of proteins using a neural embryonic stem cell model. J Neurol Sci. 229-230:261-7), with the exception that no cysteine alkylation was performed prior to iodination. Briefly, microdissected samples on LCM caps stored at −80° C. were solubilised in 8 M Urea, 4% CHAPS, 0.1 M Tris pH 7.4 at 25° C., followed by incubation of the sample at room temperature for 30 min with shaking at 1000 rpm in a Thermomixer comfirt (Eppendorf). The samples were centrifuges for 5 min at 12000 rcf and 25° C., and the soluble extracts were collected by removing the supernatant. Volumes were adjusted to 20 μL, and aliquots were taken to construct pooled samples of three tumours per pool (see above). Aliquots of these were each iodinated by either ¹²⁵I or ¹³¹I, respectively, using approximately 6 MBq of each isotope per 3.6 μg pooled sample aliquot under identical chemical conditions in a reaction volume of 25 μL by the iodogen method as described³¹. Radioactive iodine was purchased from Amersham Biosciences (Freiburg). 2D-PAGE was per-formed using 18 cm commercial immobilised pH gradients (IPGs) in serial 54 cm IPG-IEF over pH 4-9 (pH 4-5; pH 5-6; pH 6-9) that were run in the SDS-PAGE dimension as 3×18 cm IPGs in a Hoefer ISO-DALT.

TABLE 1

| ER+ pools | T378 | T392 | T460 | T464 | T288 | T711 | T712 | T425 |
|---|---|---|---|---|---|---|---|---|
| ER+1 | + | + | | | | | | |
| ER+2 | | | + | + | | | | |
| ER+3 | | | | | + | + | | |
| ER+4 | | | | | | | + | + |
| ER− pools | T433 | T443 | T469 | T470 | T531 | T558 | T623 | T640 |
| ER−1 | + | + | | | | | | |
| ER−2 | | | + | + | | | | |
| ER−3 | | | | | + | + | | |
| ER−4 | | | | | | | + | + |

TABLE 2

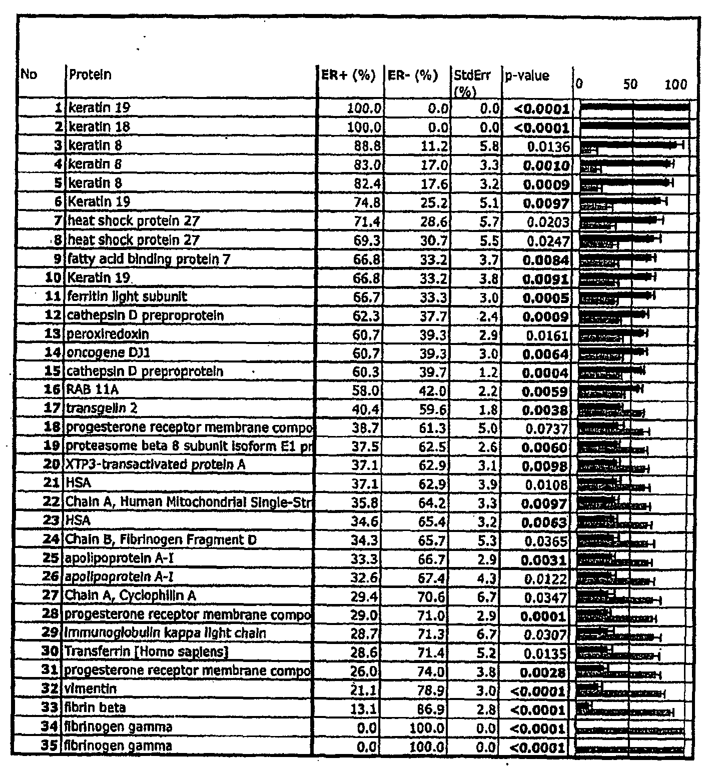

TABLE 3

| Spot | exp. | | PMF | | |
|---|---|---|---|---|---|
| No | PI | MW | Score | AccNo | Description |
| 01 | 4.9 | 41000 | 204 | gi\|34783124 | Keratin 19 [Homo sapiens] |
| 02 | 5.3 | 45000 | 80 | gi\|4557888 | keratin 18; cytokeratin 18 [Homo sapiens] |
| 05 | 5.4 | 52000 | 218 | gi\|4504919 | keratin 8; cytokeratin 8; keratin, type II cytoskeletal 8 |
| 04 | 5.4 | 55000 | 419 | gi\|4504919 | keratin 8; cytokeratin 8; keratin, type II cytoskeletal 8 |
| 05 | 5.5 | 51000 | 214 | gi\|4504919 | keratin 8; cytokeratin 8; keratin, type II cytosketelal 8 |
| 06 | 5.0 | 42000 | 395 | gi\|34783124 | Keratin 19 [Homo sapiens] |
| 07 | 5.8 | 26000 | 173 | gi\|662841 | heat shock protein 27 [Homo sapiens] |
| 08 | 5.5 | 26000 | 125 | gi\|662841 | heat shock protein 27 [Homo sapiens] |
| 09 | 5.2 | 13000 | 116 | gi\|4557585 | fatty acid binding protein 7, brain; mammary-derived growth |
| | | | | | inhibitor-related [Homo sapiens] |
| 10 | 5.0 | 43000 | 407 | gi\|34783124 | Keratin 19 [Homo sapiens] |
| 11 | 5.4 | 19000 | 113 | gi\|20149498 | ferritin, light polypeptide; ferritin light polypeptide-like 3 |
| 12 | 5.2 | 29000 | 98 | gi\|4503143 | cathepsin D preproprotein [Homo sapiens] |
| 13 | 5.4 | 21000 | 151 | gi\|33188452 | peroxiredoxin 2 isoform b; thioredoxin-dependent peroxide |
| | | | | | reductase 1; thiol-specific antioxidant 1; natural killer- |
| | | | | | enhancing factor B; thioredoxin peroxidase 1; torin |
| 14 | 5.9 | 23000 | 115 | gi\|31543380 | RNA-binding protein regulatory subunit; oncogene DJ1 |
| 15 | 5.5 | 29000 | 76 | gi\|4503143 | cathepsin D preproprotein [Homo sapiens] |
| 16 | 5.8 | 24000 | 109 | gi\|4758984 | Ras-related protein Rab-11A; RAB 11A, |
| 17 | 7.4 | 20000 | 139 | gi\|4507357 | transgelin 2; SM22-alpha homolog [Homo sapiens] |
| 18 | 4.5 | 22000 | 107 | gi\|5729875 | progesterone receptor membrane component 1 |
| 19 | 7.1 | 23000 | 68 | gi\|4758970 | proteasome beta 8 subunit isoform E1 proprotein; |
| 20 | 4.9 | 19000 | 94 | gi\|1312910 | XTP3-transactivated protein A [Homo sapiens] |
| 21 | 5.6 | 71000 | 144 | gi\|6013427 | serum albumin precursor, human serum albumin |
| 22 | 7.4 | 15000 | 105 | gi\|2624694 | Chain A, Human Mitochondrial Single-Stranded Dna |
| | | | | | Binding Protein |
| 23 | 5.7 | 71000 | 128 | gi\|6013427 | serum albumin precursor [Homo sapiens] |
| 24 | 5.6 | 40000 | 70 | gi\|2781208 | Chain B, Crystal Structure Of Fibrinogen Fragment D |
| 25 | 5.2 | 24000 | 210 | gi\|4557321 | apolipoprotein A-I precursor [Homo sapiens] |
| 26 | 5.2 | 24000 | 99 | gi\|4557321 | apolipoprotein A-I precursor [Homo sapiens] |
| 27 | 7.0 | 16000 | 85 | gi\|1633054 | Chain A, Cyclophilin A Complexed With Dipeptide Gly-Pro |
| 28 | 4.8 | 22000 | 95 | gi\|5729875 | progesterone receptor membrane component 1; |
| 29 | 7.3 | 25000 | 77 | gi\|21669399 | immunoglobulin kappa light chain VLJ region |
| 30 | 6.0 | 80000 | 99 | gi\|37747855 | Transferrin [Homo sapiens] |
| 31 | 4.5 | 22000 | 110 | gi\|5729875 | progesterone receptor membrane component 1; progesterone |
| | | | | | binding protein [Homo sapiens] |
| 32 | 4.9 | 45000 | 150 | gi\|4507895 | vimentin [Homo sapiens] |
| 33 | 6.3 | 57000 | 165 | gi\|223002 | fibrin beta |
| 34 | 5.4 | 51000 | 105 | gi\|71827 | fibrinogen gamma-A chain precursor [validated] - human |
| 35 | 5.3 | 49000 | 66 | gi\|71827 | fibrinogen gamma-A chain precursor [validated] - human |

TABLE 4

| theor | | Exp. | | | PMF | |
|---|---|---|---|---|---|---|
| MW | pI | PI | MW | AccNo | Score | Description |
| 45587 | 4.85 | 5.0 | 43000 | gi\|34783124 | 407 | Keratin 19 [Homo sapiens] |
| | | 5.0 | 42000 | | 395 | |
| | | 5.0 | 42000 | | 424 | |
| | | 4.9 | 41000 | | 204 | |
| 55874 | 5.38 | 5.3 | 56000 | gi\|39645331 | 154 | KRT8 protein. |
| 53671 | 5.26 | 5.1 | 49000 | gi\|4504919 | 102 | keratin 8; cytokeratin 8; keratin, type II cytoskeletal 8 |
| | | 5.2 | 49000 | | 215 | [Homo sapiens] |
| | | 5.4 | 55000 | | 419 | |
| 22667 | 8.24 | 5.4 | 26000 | gi\|662841 | 123 | heat shock protein 27 [Homo sapiens] |
| | | 5.5 | 26000 | | 125 | |
| 22638 | 8.96 | 7.8 | 22000 | gi\|4507359 | 95 | transgelin; SM22-alpha [Homo sapiens] |
| | | 8.5 | 21000 | | 137 | |
| 18578 | 7.97 | 6.8 | 16000 | gi\|1633054 | 87 | Chain A, Cyclophilin A |
| | | 7.4 | 15000 | | 81 | |
| | | 7.0 | 16000 | | 85 | |
| 22012 | 4.30 | 4.6 | 22000 | gi\|5729875 | 95 | progesterone receptor membrane component 1; |
| | | 4.5 | 22000 | | 110 | progesterone binding protein; Hpr6.6[Homo sapiens] |
| | | 4.5 | 22000 | | 107 | |
| 53830 | 4.77 | 4.8 | 47000 | gi\|4507895 | 336 | vimentin [Homo sapiens] |
| | | 5.0 | 56000 | | 531 | |
| | | 4.9 | 45000 | | 150 | |
| 58017 | 8.38 | 6.1 | 59000 | gi\|399492 | 79 | Fibrinogen beta chain precursor [Contains: Fibrino- |
| | | 6.1 | 57000 | | 85 | peptide B]. Chain B, Crystal Structure Of Fibrinogen |
| | | 6.8 | 55000 | | 66 | Fragment D |
| | | 7.1 | 57000 | | 88 | |
| 39041 | 6.06 | 5.6 | 40000 | gi\|2781208 | 70 | |

TABLE 5

| PR+ pools | T698 | T851 | T876 | T259 | T533 | T630 | T415 | T595 | T764 | T794 | T816 | T869 |
|---|---|---|---|---|---|---|---|---|---|---|---|---|
| PR+1 | + | + | + | | | | | | | | | |
| PR+2 | | | | + | + | + | | | | | | |
| PR+3 | | | | | | | + | + | + | | | |
| PR+4 | | | | | | | | | | + | + | + |

| PR− pools | H623 | H69 | T680 | H579 | T382 | T894 | T425 | T413 | T802 | H574 | T2 | T3228 |
|---|---|---|---|---|---|---|---|---|---|---|---|---|
| PR−1 | + | + | + | | | | | | | | | |
| PR−2 | | | | + | + | + | | | | | | |
| PR−3 | | | | | | | + | + | + | | | |
| PR−4 | | | | | | | | | | + | + | + |

TABLE 6

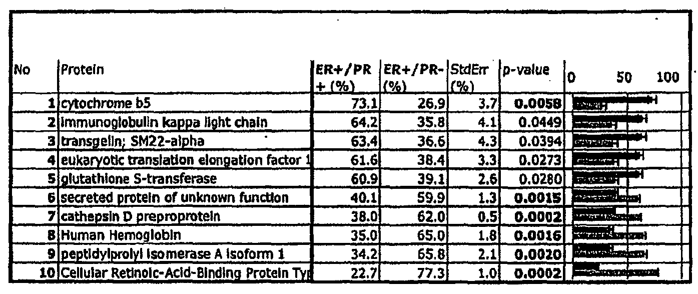

TABLE 7

| | exper. | | | | PMF | theor | |
|---|---|---|---|---|---|---|---|
| No | pI | MW | AccNo | Description | Score | pI | MW |
| 1 | 4.7 | 16000 | gi\|353818 | cytochrome b5 | 102 | 4.8 | 11130 |
| 2 | 5.9 | 25000 | gi\|21669479 | immunoglobulin kappa light chain VLJ region [Homo sapiens] | 73 | 6.7 | 29807 |
| 3 | 6.4 | 18000 | gi\|4507359 | transgelin; SM22-alpha [Homo sapiens] | 113 | 8.9 | 22638 |
| 4 | 4.9 | 34000 | gi\|25453472 | eukaryotic translation elongation factor 1 delta isoform 2; guanine | 88 | 4.6 | 31457 |
| | | | | nucleotide exchange protein [Homo sapiens] | | | |
| 5 | 5.7 | 22000 | gi\|2204207 | glutathione S-transferase [Homo sapiens] | 84 | 5.3 | 24075 |
| 6 | 4.8 | 16000 | gi\|7019545 | secreted protein of unknown function [Homo sapiens] | 89 | 5.3 | 18845 |
| 7 | 5.3 | 28000 | gi\|4503143 | cathepsin D preproprotein [Homo sapiens] | 103 | 6.5 | 46117 |
| 8 | 7.0 | 12000 | gi\|1942686 | Chain B, Human Hemoglobin | 104 | 7.3 | 15988 |
| 9 | 7.0 | 15000 | gi\|10863927 | peptidylprolyl isomerase A isoform 1; cyclophilin A; peptidyl-prolyl | 113 | 8.0 | 18709 |
| | | | | cis-trans isomerase A; T cell cyclophilin; rotamase; cyclosporin A- | | | |
| | | | | binding protein [Homo sapiens] | | | |
| 10 | 5.4 | 13000 | gi\|3318697 | Chain A, Apo-Cellular Retinoic Acid Binding Protein II | 121 | 4.8 | 16058 |

TABLE 8

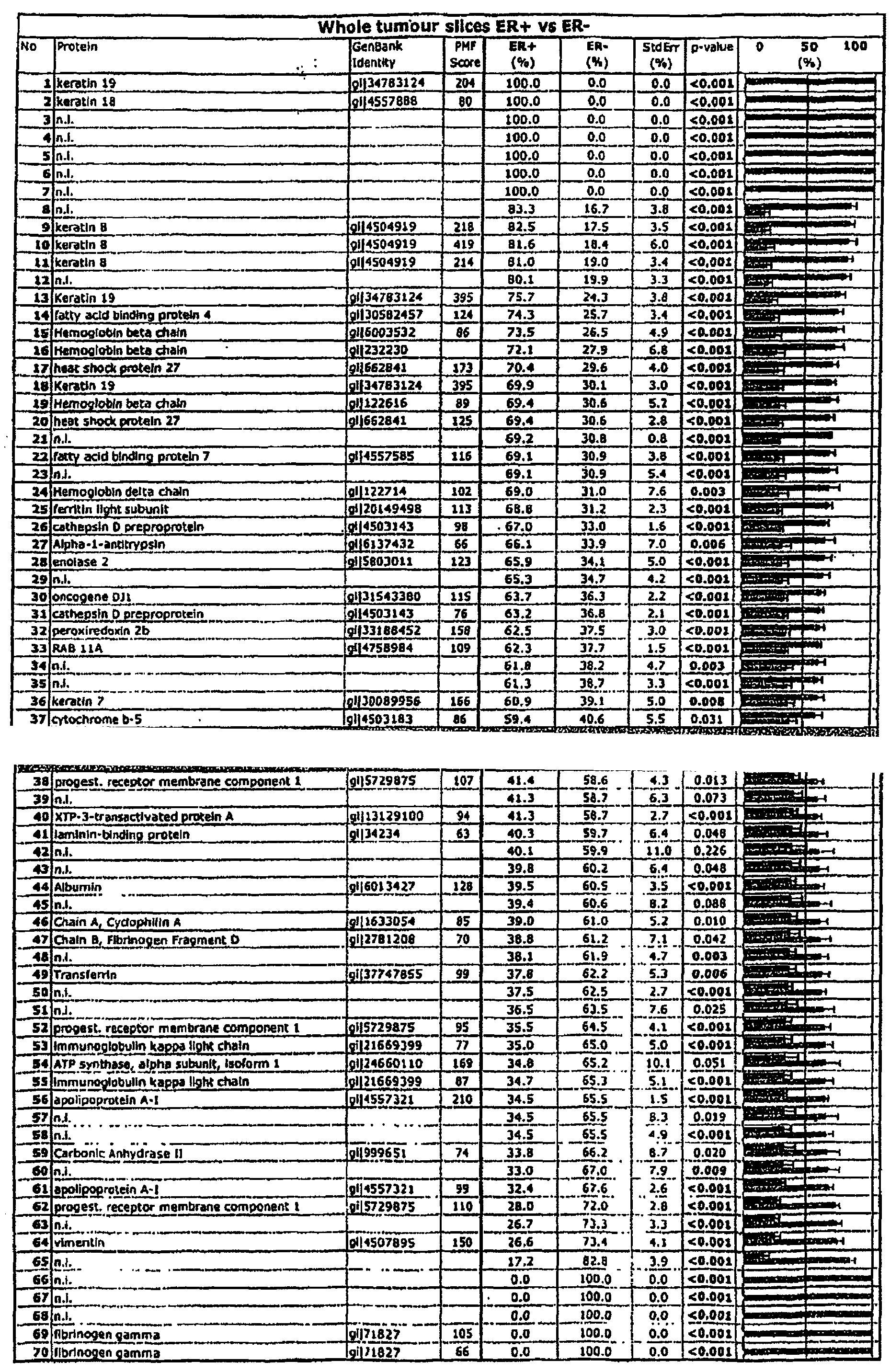

TABLE 9

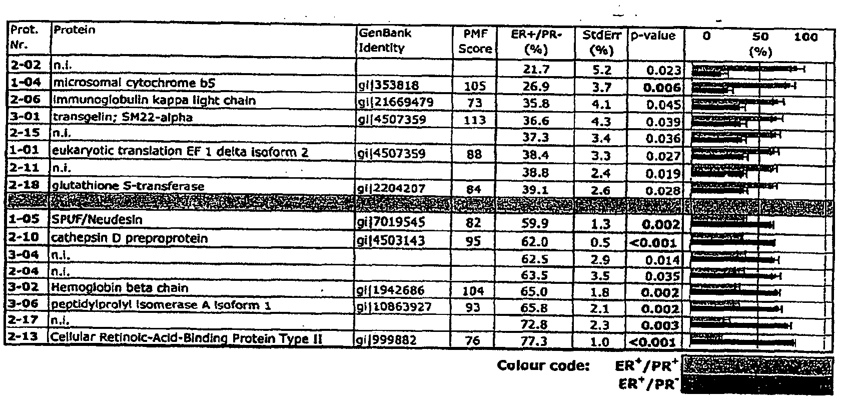

TABLE 10

| Experimental | | | LCM- | | | Lymph | | | | Her2/ | | Preparative |
|---|---|---|---|---|---|---|---|---|---|---|---|---|
| variable | | Tumor | Shots | RNA | Tumor | node | | ER | PR | neu- | Age of | Slices |
| designation | Pools | number | (30 μm) | Quality | status | status | Grade | status | status | Status | patient | (10 μm) |
| ER+/PR− | PR−1 | H 623 | 10 000 | ok | pT2 | pN0 | 2 | 12 | 0 | 0 | 56 | 35 |
| | | H 69 | 10 000 | ok | PT2 | pN0 | 2 | 12 | 0 | 0 | 59 | 35 |
| | | T680 | 10 000 | ok | pT2 | pN0 | 2 | 12 | 0 | 2* | 38 | 35 |
| | PR−2 | H 579 | 10 000 | ok | pT1 | pN0 | 3 | 12 | 0 | 0 | 63 | 45 |
| | | T382 | 10 000 | ok | pT1 | pN0 | 2 | 12 | 0 | 0 | 57 | 55 |
| | | T894 I | 10 000 | ok | pT1 | pN0 | 2 | 12 | 0 | 0 | 65 | 45 |
| | PR−3 | T425 III | 10 000 | ok | pT2 | pN1 | 2 | 12 | 0 | 0 | 78 | 45 |
| | | T413 | 10 000 | ok | pT3 | pN1 | 2 | 12 | 0 | 1 | 60 | 35 |
| | | T802 | 10 000 | ok | pT2 | pN1 | 2-3 | 12 | 0 | 0 | 76 | 35 |
| | PR−4 | H 574 II | 10 000 | ok | pT2 | pN0 | 3 | 12 | 0 | 0 | 76 | 55 |
| | | T2 | 10 000 | ok | pT2 | pNx | 2 | 12 | 0 | 1 | 77 | 35 |
| | | T228 | 10 000 | ok | pT3 | pN1 | 3 | 12 | 0 | 0 | 62 | 45 |
| ER+/PR+ | PR+1 | T698 | 10 000 | ok | pT2 | pN0 | 2 | 12 | 12 | 0 | 65 | 35 |
| | | T851 | 10 000 | ok | pT1 | pN0 | 2 | 12 | 9 | 0 | 78 | 35 |
| | | T876 | 10 000 | ok | pT2 | pN0 | 2 | 12 | 9 | 0 | 70 | 35 |
| | PR+2 | T259 | 10 000 | ok | pT1 | pN0 | 2 | 12 | 9 | 0 | 70 | 35 |
| | | T533 | 10 000 | ok | pT2 | pN1 | 2 | 12 | 12 | 1 | 65 | 35 |
| | | T630 | 10 000 | ok | pT1 | pN0 | 2 | 12 | 12 | 0 | 70 | 35 |
| | PR+3 | T415 | 10 000 | ok | pT2 | pN1 | 2 | 12 | 8 | 0 | 67 | 35 |
| | | T595 | 10 000 | ok | pT2 | pN1 | 2 | 12 | 8 | 0 | 73 | 35 |
| | | T764 | 10 000 | ok | pT2 | pN1 | 2 | 12 | 12 | 0 | 42 | 35 |
| | PR+4 | T794 | 10 000 | ok | pT1 | pN2 | 2 | 12 | 12 | 1 | 65 | 35 |
| | | T816 | 10 000 | ok | pT1 | pNX | 2 | 12 | 6 | 0 | 66 | 35 |
| | | T869 | 10 000 | ok | pT1 | pN3 | 2 | 12 | 12 | 0 | 64 | 35 |

1-24. (canceled)

25. Method for diagnosing or treating cancer, especially cancer that is associated with poorer disease-free and overall survival probability, wherein at least one isoform of progesterone receptor membrane component 1 (Hpr6.6 protein) is used as diagnostic marker and/or therapeutic target.

26. Method according to claim 25 wherein at least two isoforms are used as diagnostic markers and/or therapeutic targets for said cancer.

27. Method according to claim 25 wherein said at least one isoform has a significantly increased abundance in cancer cells of said cancer.

28. Method according to claim 25 wherein said isoforms of progesterone receptor membrane component 1 (Hpr6.6) differ from each other in their phosphorylation status.

29. Method according to claim 25 wherein said cancer is breast cancer.

30. Method according to claim 29 wherein breast cancer cells of said breast cancer are negative for the estrogen receptor (ER−).

31. Method according to claim 29 wherein breast cancer cells of said breast cancer are negative for the progesterone receptor (PR−).
